(19) United States
(12) Patent Application Publication
(10) Pub. No.: US 2024/0003888 A1
(43) Pub. Date: Jan. 4, 2024

(54) METHODS FOR IDENTIFYING DRUGGABLE TARGETS AND TREATING CANCER

(71) Applicant: GUARDANT HEALTH, INC., Palo Alto, CA (US)

(72) Inventor: Scott R. Bortner, Redwood City, CA (US)

(21) Appl. No.: 18/319,344

(22) Filed: May 17, 2023

Related U.S. Application Data
(60) Provisional application No. 63/364,841, filed on May 17, 2022.

(51) Int. Cl. G01N 33/574

(52) U.S. Cl. CPC G01N 33/57484; G01N 2500/20

(57) ABSTRACT

This disclosure provides methods, sets, and kits for predicting and identifying druggable targets for treating cancer. This disclosure further provides methods of treating cancer.

### CROSS-REFERENCE

This application claims the benefit under 5 U.S.C. § 119(e) of U.S. Provisional Application No. 63/364,841, filed May 17, 2022.

### BACKGROUND

Early detection of cancer is an ongoing challenge, limited for decades by diagnostic methods that relied on solid tissue biopsies. Recently, liquid biopsies have emerged as a promising method for non-invasive diagnosis or monitoring of cancer. There remains, however, a need in the art for improved methods that streamline the diagnosis and treatment selection process when using liquid biopsy samples. The present disclosure addresses this long-felt need by providing improved methods and sets of tests for diagnosing patients with cancer, predicting and confirming druggable targets, selecting appropriate treatments, and/or administering appropriate treatments to cancer patients in a streamlined and reduced-cost manner.

### SUMMARY

The methods and sets of tests described herein provide for improved detection and/or treatment of cancer. In some aspects, the methods comprise identifying one or more druggable targets for treating cancer in a subject. In some aspects, the methods comprise predicting and confirming a druggable target for treating cancer in a subject. In some aspects, the methods comprise selecting a targeted cancer diagnostic test for a subject. In some aspects, the methods comprise treating cancer in a subject. In some aspects, the present disclosure provides a set of a first and second test for identifying one or more druggable targets for treating cancer in a subject. In some aspects, the present disclosure provides a set of a first and second test for predicting and assessing one or more druggable targets for treating cancer in a subject. Assessing includes the detecting of the presence (or absence) of genetic variants that are useful determining the selection of specific therapeutic agent or agents.

In various aspects, the present disclosure provides a method of identifying one or more druggable targets for treating cancer in a subject, wherein the method comprises performing or having performed a first test on a liquid biopsy sample of the subject to identify that the subject has a cancer, and performing a second test, wherein the second test assesses genetic variants in the subject to identify the presence of one or more druggable targets. In some embodiments, the second test is performed on a liquid biopsy sample of the subject. The first and second tests may be performed on different liquid biopsy samples of the subject. Alternatively, the first and second tests may be performed on the same liquid biopsy sample.

In various aspects, the present disclosure provides a method of treating cancer in a subject, wherein the method comprises performing or having performed a first test on a liquid biopsy sample of the subject to identify that the subject has a cancer; performing or having performed a second test, wherein the second test assesses genetic variants in the subject to identify the presence of one or more druggable targets; and administering to the subject an effective amount of a drug that targets at least one of the identified druggable targets. In some embodiments, the second test is or has been performed on a liquid biopsy sample of the subject. The first and second tests may be performed on different liquid biopsy samples of the subject. Alternatively, the first and second tests may be performed on the same liquid biopsy sample. In embodiments in which two or more samples are tested, the samples may be collected at the same time or at different time points. In some embodiments, a single sample may be split into separate portions for running different tests on each portion.

In various aspects, the present disclosure provides a method of selecting a targeted cancer diagnostic test for a subject, wherein the method comprises performing or having performed a first test on a liquid biopsy sample of the subject to identify that the subject has a cancer, wherein the first test produces a profile that is predictive of one or more druggable targets in the subject; and selecting a second test as the targeted cancer diagnostic test, wherein the second test assesses the presence or absence of the one or more druggable targets predicted by the profile.

In various aspects, the present disclosure provides a method of predicting and confirming (or detecting) a druggable target for treating cancer in a subject, wherein the method comprises performing or having performed a first test on a liquid biopsy sample of the subject to identify that the subject has a cancer, wherein the first test produces a profile that is predictive, e.g., increased likelihood having, of one or more druggable targets in the subject; selecting a second test, wherein the second test assesses the presence or absence of the one or more druggable targets; and performing the second test, wherein the second test confirms (e.g., detects) the presence of at least one of the one or more druggable targets predicted by the profile generated by the first test. In some embodiments, the second test is performed on a liquid biopsy sample of the subject. The first and second tests may be performed on different liquid biopsy samples of the subject. Alternatively, the first and second tests may be performed on the same liquid biopsy sample.

In various aspects, the present disclosure provides a method of treating cancer in a subject, wherein the method comprises performing or having performed a first test on a liquid biopsy sample of the subject to identify that the subject has a cancer, wherein the first test produces a profile that is predictive of one or more druggable targets in the subject; selecting or having selected a second test, wherein the second test assesses the presence or absence of the one or more druggable targets; performing or having performed the second test, wherein the second test confirms the presence of at least one of the one or more druggable targets predicted by the profile generated by the first test; and administering to the subject an effective amount of a drug that targets the at least one confirmed druggable target. In some embodiments, the second test is or has been performed on a liquid biopsy sample of the subject. The first and second tests may be performed on different liquid biopsy samples of the subject. Alternatively, the first and second tests may be performed on the same liquid biopsy sample.

In various aspects, the present disclosure provides a method of selecting a targeted cancer diagnostic test for a subject, wherein the method comprises performing or having performed a first test on a liquid biopsy sample of the subject to identify that the subject has a cancer, wherein the first test produces a first or second profile, optionally a first or second epigenetic profile, wherein the first profile is predictive of a first panel of one or more druggable targets in the subject and the second profile is predictive of a second panel of one or more druggable targets in the subject; and selecting a second test as the targeted cancer diagnostic test. In some embodiments, if the first test produces the first profile, the second test assesses the presence or absence of the first panel of one or more druggable targets predicted by the first profile; and if the first test produces the second profile, the second test assesses the presence or absence of the second panel of one or more druggable targets predicted by the first profile. In various aspects, the observation of certain profile will suggest that one panel is more predictive than another panel, thereby providing guidance in the selection of a more predictive panel.

In various aspects, the present disclosure provides a method of predicting and confirming a druggable target for treating cancer in a subject, wherein the method comprises performing or having performed a first test on a liquid biopsy sample of the subject to identify that the subject has a cancer, wherein the first test produces a first or second profile, optionally a first or second epigenetic profile, wherein the first profile is predictive of a first panel of one or more druggable targets in the subject and the second profile is predictive of a second panel of one or more druggable targets in the subject; selecting or having selected a second test as the targeted cancer diagnostic test, wherein if the first test produces the first profile, the second test assesses the presence or absence of the first panel of one or more druggable targets; and if the first test produces the second profile, the second test assesses the presence or absence of the second panel of one or more druggable targets; and performing the second test, wherein the second test confirms the presence of at least one of the one or more druggable targets. In some embodiments, the second test is performed on a liquid biopsy sample of the subject. The first and second tests may be performed on different liquid biopsy samples of the subject. Alternatively, the first and second tests may be performed on the same liquid biopsy sample.

In various aspects, the present disclosure provides a method of treating cancer in a subject, wherein the method comprises performing or having performed a first test on a liquid biopsy sample of the subject to identify that the subject has a cancer, wherein the first test produces a first or second profile, optionally a first or second epigenetic profile, wherein the first profile is predictive of a first panel of one or more druggable targets in the subject and the second profile is predictive of a second panel of one or more druggable targets in the subject; selecting or having selected a second test as the targeted cancer diagnostic test, wherein if the first test produces the first profile, the second test assesses the presence or absence of the first panel of one or more druggable targets; and if the first test produces the second profile, the second test assesses the presence or absence of the second panel of one or more druggable targets; and performing or having performed the second test, wherein the second test confirms the presence of at least one of the one or more druggable targets; and administering to the subject an effective amount of a drug that targets the at least one confirmed druggable target. In some embodiments, the second test is or has been performed on a liquid biopsy sample of the subject. The first and second test may be performed on different liquid biopsy samples of the subject. Alternatively, the first and second tests may be performed on the same liquid biopsy sample.

In some embodiments, the first test comprises a methylation assay, a nucleic acid assay (e.g., a genetic screen a protein assay, a protein post-translational modification assay, a fragmentomics assay, a metabolomics assay, an RNA assay (e.g., a microRNA (miRNA) assay), a microbiome assay, an assay of one or more immune cell populations, or a combination thereof. In some embodiments, the first test comprises a methylation assay, a nucleic acid assay (e.g., a genetic screen), a protein assay, a fragmentomics assay, or a combination thereof. In some embodiments, the first test comprises a methylation assay, a nucleic acid assay (e.g., a genetic screen), a protein assay, and a fragmentomics assay. In some embodiments, the first test comprises a methylation assay.

In some embodiments, the profile produced by the first test comprises data on methylation state, chromatin compaction, histone modification, fragmentation patterns, topology, other epigenetic data, nucleic acid sequence, nucleic acid expression, protein translation, protein sequence, protein post-translational modification (e.g., glycosylation), metabolite presence, microbiome composition, immune state, or a combination thereof. In some embodiments, the profile comprises data on methylation state, chromatin compaction, histone modification, fragmentation patterns, topology, other epigenetic data, or a combination thereof.

In some embodiments, the first test comprises multiple assays. In some embodiments, the profile produced by the first test comprises data from multiple assays. In other embodiments, the profile comprises data from a single assay of the multiple assays.

In some embodiments, the first test comprises a single assay and the profile produced by the first test comprises data from the single assay.

In some embodiments, a second test is selected and/or performed as a result of the profile produced by the first test. In some embodiments, the second test comprises a test for one or more genetic variants. In some embodiments, the second test identifies the presence of, assesses the presence or absence of, and/or confirms the presence of at least one of the one or more druggable targets. In some embodiments, the one or more druggable targets comprises the one or more genetic variants. In some embodiments, the one or more druggable targets comprises a ribonucleic acid expression product of the one or more genetic variants. In some embodiments, the one or more druggable targets comprises a peptide or protein encoded by the one or more genetic variants. In some embodiments, the one or more druggable targets comprises a nucleic acid, peptide, or protein that shares a signaling pathway with the one or more genetic variants.

In some embodiments, the liquid biopsy sample comprises a blood sample. In some embodiments, the blood sample comprises one or more components of whole blood. In some embodiments, the blood sample comprises serum. In some embodiments, the blood sample comprises plasma. In some embodiments, the liquid biopsy sample comprises cell-free DNA.

In various aspects, the present disclosure provides a set of a first and second test for identifying one or more druggable targets for treating cancer in a subject, wherein the set comprises a first liquid biopsy test that identifies if the subject has a cancer and a second liquid biopsy test that assesses genetic variants in the subject to identify the presence of one or more druggable targets.

In various aspects, the present disclosure provides a set of a first and second test for predicting and assessing one or more druggable targets for treating cancer in a subject, wherein the set comprises a first liquid biopsy test that produces a profile that is predictive of one or more druggable targets in the subject and a second liquid biopsy test that assesses the presence or absence of the one or more druggable targets predicted by the profile.

In various aspects, the present disclosure provides a set of a first and second test for predicting and assessing one or more druggable targets for treating cancer in a subject, wherein the set comprises a first liquid biopsy test that produces a first or second profile, optionally a first or second epigenetic profile, wherein the first profile is predictive of a first panel of one or more druggable targets in the subject and the second profile is predictive of a second panel of one or more druggable targets in the subject; and a second liquid biopsy test. In some embodiments, if the first liquid biopsy test produces the first profile, the second liquid biopsy test assesses the presence or absence of the first panel of one or more druggable targets; and if the first liquid biopsy test produces the second profile, the second liquid biopsy test assesses the presence or absence of the second panel of one or more druggable targets predicted by the first profile.

In some embodiments, the set comprises a first liquid biopsy test that comprises a methylation assay, a nucleic acid assay (e.g., a genetic screen), a protein assay, a protein post-translational modification assay, a fragmentomics assay, a metabolomics assay, an RNA assay (e.g., a microRNA (miRNA) assay), a microbiome assay, an assay of one or more immune cell populations, or a combination thereof. In some embodiments, the set comprises a first liquid biopsy test that comprises a methylation assay, a nucleic acid assay (e.g., a genetic screen), a protein assay, a fragmentomics assay, or a combination thereof. In some embodiments, the set comprises a first test that comprises a methylation assay, a nucleic acid assay (e.g., a genetic screen), a protein assay, and a fragmentomics assay. In some embodiments, the set comprises a first liquid biopsy test that comprises a methylation assay.

In some embodiments, the profile produced by the first liquid biopsy test of the set comprises data on methylation state, chromatin compaction, histone modification, fragmentation patterns, topology, other epigenetic data, nucleic acid sequence, nucleic acid expression, protein translation, protein sequence, protein post-translational modification (e.g., glycosylation), metabolite presence, microbiome composition, immune state, or a combination thereof. In some embodiments, the profile produced by the first liquid biopsy test of the set comprises data on methylation state, chromatin compaction, histone modification, fragmentation patterns, topology, other epigenetic data, or a combination thereof.

In some embodiments, the set comprises a first liquid biopsy test that comprises multiple assays. In some embodiments, the profile produced by the first liquid biopsy test of the set comprises data from multiple assays. In other embodiments, the profile comprises data from a single assay of the multiple assays.

In some embodiments, the set comprises a first liquid biopsy test that comprises a single assay and the profile produced by the first liquid biopsy test comprises data from the single assay.

In some embodiments, the set comprises a second test that is selected and/or performed as a result of the profile produced by the first test of the set. In some embodiments, the second test of the set comprises a test for one or more genetic variants. In some embodiments, the second test of the set identifies the presence of, assesses the presence or absence of, or confirms the presence of at least one of the one or more druggable targets. In some embodiments, the one or more druggable targets comprises the one or more genetic variants. In some embodiments, the one or more druggable targets comprises a ribonucleic acid expression product of the one or more genetic variants. In some embodiments, the one or more druggable targets comprises a peptide or protein encoded by the one or more genetic variants. In some embodiments, the one or more druggable targets comprises a nucleic acid, peptide, or protein that shares a signaling pathway with the one or more genetic variants.

In some embodiments, the first liquid biopsy test of the set is configured to be performed on a blood sample. In some embodiments, the second liquid biopsy test of the set is configured to be performed on a blood sample. In some embodiments, the first and second liquid biopsy tests of the set are configured to be performed on a blood sample.

In some embodiments, the first liquid biopsy test of the set is configured to be performed on cell-free DNA. In some embodiments, the second liquid biopsy test is configured to be performed on cell-free DNA. In some embodiments, the first and second liquid biopsy tests are configured to be performed on cell-free DNA.

By using the profile observed in the first test to select the specific set of druggable targets analyzed in the second test cost savings may be obtained by reducing the amount of target sequence information that is needs to be analyzed.

Studies determining correlations between and epigenetic profiles and genetic variations may be used in the selection of genome target regions for genetic analysis for obtaining drug selection information.

Other features, objects, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### DETAILED DESCRIPTION

The present disclosure provides methods and sets of tests for improved detection and/or treatment of cancer wherein the method or sets of tests are performed on a liquid biopsy sample.

### Definitions

As used herein, “druggable target” means a biological target that is known to be or predicted to be capable of therapeutic modulation. Certain examples of druggable targets and drugs capable of modulating druggable targets are provided in this disclosure.

As used herein, the singular terms “a,” “an,” and “the” include the plural reference unless the context clearly indicates otherwise. Further, unless otherwise required by context, singular terms shall include pluralities and plural terms shall include the singular.

The phrases “and/or,” “at least one,” and “one or more,” as used herein, each mean one, all, or any sub-combination of the elements in a list of elements. Thus, as a non-limiting example, “A, B, and/or C,” encompasses any of: A alone; B alone; C alone; A and B without C; A and C without B; B and C without A; and A, B, and C.

As used herein, “a combination thereof” means any two or more of the elements in a list of elements.

Throughout this disclosure, the words “have” and “comprise,” or variations such as “has,” “having,” “comprises,” or “comprising,” will be understood to imply the inclusion of a stated element or group of elements but not the exclusion of any other element or group of elements. Although a number of documents are cited herein, this citation does not constitute an admission that any of these documents forms part of the common general knowledge in the art. All publications and other references mentioned herein are incorporated by reference in their entirety. In the event of a conflict between an incorporated reference and the present disclosure, the present disclosure controls.

### Liquid Biopsies

Liquid biopsies are a non-invasive method of using a non-solid biological sample. In some embodiments, the liquid biopsy sample may be, e.g., blood, cerebrospinal fluid, urine, saliva, sputum, pleural effusions, and/or amniotic fluid. The liquid biopsy sample, for instance a blood sample, can contain a range of cell types (e.g., immune cells and/or circulating tumor cells) and cell products (e.g., DNA, RNA, peptide, and/or protein). In some embodiments, the cell products emanate from one or multiple tumor sites around the body. Exemplary biopsy samples include circulating tumor cells (CTCs), circulating nucleic acids (including circulating cell-free tumor DNA (ctDNA) and cell-free DNA (cfDNA), as well as cell-free RNAs (e.g., mRNAs, long non-coding RNAs, microRNAs, and/or circular RNAs)), extracellular vesicles, tumor-secreted vesicles (e.g., exosomes, oncosomes, and/or apoptotic bodies), tumor-educated platelets, proteins, and metabolites (e.g., branched-chain amino acids (BCAAs)). In some embodiments, these samples provide information about features of primary tumors or metastases, including site of origin, genomic mutations, and copy number alterations. In some embodiments, these samples provide information about one or more of the transcriptome, epigenome, proteome, and metabolome. Some further details regarding certain liquid biopsies and their use in diagnostics are known in the art. See, e.g., Heitzer et al. “Current and future perspectives of liquid biopsies in genomics-driven oncology.” Nat Rev Genet 20, 71-88 (2019); Kilgour et al. “Liquid biopsy-based biomarkers of treatment response and resistance.” Cancer Cell 37:4, 485-495 (2020); Ignatiadis et al. “Liquid biopsy enters the clinic-implementation issues and future challenges.” Nat Rev Clin Onc 18, 297-312 (2021); and Wan et al. “Liquid biopsies come of age: towards implementation of circulating tumour DNA.” Nat Rev Cancer 17, 223-238 (2017).

In some embodiments, the liquid biopsy test detects or diagnoses a cancer. In some embodiments, the liquid biopsy test does not detect a cancer. In some embodiments, the liquid biopsy test detects a cancer's stage. In some embodiments, the liquid biopsy test detects an early-stage cancer. In some embodiments, the liquid biopsy test detects recurrent cancer following curative-intent treatment of a primary tumor. In some embodiments, the liquid biopsy test detects metastatic cancer.

In some embodiments, the liquid biopsy test guides the selection of an appropriate treatment for a cancer. In some embodiments, the liquid biopsy test guides the selection of an appropriate second test from a panel of possible second tests for identifying one or more appropriate and/or unsuitable treatments for a cancer. In some embodiments, an appropriate treatment is one to which the cancer is likely to be susceptible. In some embodiments, an appropriate treatment is one to which the cancer is unlikely to be resistant. In some embodiments, an appropriate treatment targets one or more biomarkers detected by a test as described herein. In some embodiments, the liquid biopsy test identifies that a cancer is resistant or is likely to be resistant to one or more potential treatments, thus identifying that those one or more potential treatments are unsuitable.

In some embodiments, the liquid biopsy sample is a blood, serum, or plasma sample. In some embodiments, the liquid biopsy sample is a serum sample. In some embodiments, the liquid biopsy sample is a plasma sample. In some embodiments, the liquid biopsy sample is a cell-free sample. Certain methods for collecting blood samples for analysis of cell-free DNA are known in the art. See, e.g., Aggarwal et al. “Strategies for the successful implementation of plasma-based NSCLC genotyping in clinical practice.” Nat Rev Clin Oncol 18, 56-62 (2020).

### Assays

As used herein, the term “assay” refers to a technique for determining one or more properties of one or more substances, e.g., a nucleic acid, a protein, a cell, a tissue, and/or an organ. An assay (e.g., a first assay or a second assay) can comprise a methylation assay, a nucleic acid assay (e.g., a genetic screen), a protein assay, a protein post-translational modification assay, a fragmentomics assay, a metabolomics assay, an RNA assay (e.g., a microRNA (miRNA) assay), a microbiome assay, an assay of one or more immune cell populations, or a combination thereof. An assay can be used to detect the properties of one or more components of a liquid biopsy, e.g., cfDNA, extracellular vesicles, proteins, metabolites, and/or circulating tumor cells. A “test” or “liquid biopsy test” may comprise one or more assays.

In some embodiments, a first liquid biopsy sample is assayed to generate a profile comprising data on methylation state, chromatin compaction, histone modification, fragmentation patterns, topology, other epigenetic data, nucleic acid sequence, nucleic acid expression, protein translation, protein sequence, protein post-translational modification (e.g., glycosylation), metabolite presence, microbiome composition, immune state, or a combination thereof.

In some embodiments, the test comprises a nucleic acid assay. In some embodiments, the nucleic acid assay comprises a DNA assay. In some embodiments, the DNA assay assesses one or more genetic variants in the subject. In some embodiments, the DNA assay generates a profile comprising data on the subject's one or more genetic variants. Any method of isolating and assessing DNA may be used. See, e.g., WO2018083467A1; WO2017181202A2; WO2019241250A1; WO2019018757A1; WO2017151524A1; WO2017151502A1; WO2016149261A1; WO2020047378A1; Alekseyev et al. “A next-generation sequencing primer—how does it work and what can it do?” Academic Pathology 5, 1-11 (2018); and McCombie et al. “Next-generation sequencing technologies.” Cold Spring Harb Perspect Med (2019).

In some embodiments, the DNA assay may comprise a sequencing technique known in the art. Exemplary sequencing techniques include targeted sequencing using PCR amplicons, hybrid-capture sequencing, targeted capture sequencing, whole-genome sequencing (WGS), shallow WGS, targeted sequencing of single-nucleotide polymorphisms, BEAMing (beads, emulsion, amplification, and magnetics), Intolex, COLD-PCR (co-amplification at lower denaturation temperature PCR), multiplex PCR, SCODA (synchronous coefficient of drag alteration), NaME-PrO (nuclease-assisted minor-allele enrichment with probe-overlap), ARMS-PCR kits for companion diagnostics (CDx), cobas EGFR, therascreen EGFR, single-cell reduced representation bisulfite sequencing, TAm-Seq (tagged amplicon deep sequencing), enhanced TAm-Seq, Safe-SeqS, exome sequencing, CAPP-Seq (cancer personalized profiling by deep sequencing), digital sequencing, TEC-seq (targeted error correction sequencing), Plasma-Seq, PARE (personalized analysis of rearranged ends), FAST-SeqS (fast aneuploidy screening test-sequencing system), mFAST-SeqS (modified fast aneuploidy screening test-sequencing system), allele-specific PCR, whole exome sequencing (WES), UroSEEK, PapSEEK, MSK-ACCESS (Memorial Sloan Kettering-Analysis of Circulating Cell-free DNA to Evaluate Somatic Status), Archer Reveal ctDNA 28 assay, FoundationACT (incorporating measurement of blood tumor mutational burden (bTMB)), FoundationOne Liquid CDx, Guardant360 CDx, Inivata InVision, OncoDNA OncoSTRAT&GO, PGDx elio plasma resolve, Resolution Bioscience Resolution ctDx, multiplexed targeted digital sequencing (TARDIS), single molecule real-time sequencing, ion semiconductor sequencing, pyrosequencing, sequencing by synthesis, sequencing by ligation, Sanger sequencing, and Droplet digital PCR (ddPCR). In some embodiments, the DNA assay is allele specific.

In some embodiments, the DNA assay assesses a cell-free DNA (cfDNA; also known as circulating DNA) sample. In some embodiments, the DNA assay generates a profile comprising data on cfDNA. In some embodiments, cfDNA is identified or quantified using a method known in the art, including DNA sequencing techniques. In some embodiments, the cfDNA sample comprises bodily fluids such as blood, whole blood, plasma, serum, urine, cerebrospinal fluid, feces, saliva, sweat, tears, pleural fluid, pericardial fluid, or peritoneal fluid of a subject. Certain exemplary cell-free nucleic acids include RNA, mitochondrial DNA, and genomic DNA. In some embodiments, the cfDNA is nucleosome-associated. In some embodiments, the cfDNA comprises DNA from healthy and/or cancerous cells. In some embodiments, cfDNA can have one or more epigenetic modifications. Certain exemplary modifications include acetylation, 5-methylation, ubiquitylation, phosphorylation, sumoylation, ribosylation, and citrullination.

In some embodiments, the DNA assay assesses circulating tumor DNA (ctDNA; also known as circulating cell-free tumor DNA). In some embodiments, the DNA assay generates a profile comprising data on ctDNA. In some embodiments, ctDNA is identified or quantified using a method known in the art, including fragment length or methylation status. See, e.g., Mardis “The emergence of cancer genomics in diagnosis and precision medicine.” Nat Cancer 2, 1263-1264 (2021). In some embodiments, ctDNA levels vary according to disease stage, metabolic tumor volume, tumor histology and/or radiological appearance of the tumors. See, e.g., Rolfo and Russo “Liquid biopsy for early stage lung cancer moves ever closer.” Nat Rev Clin Onc 17, 523-524 (2020).

In some embodiments, the nucleic acid assay comprises an RNA assay. In some embodiments, the RNA assay generates an RNA profile comprising data on RNA molecules (e.g., mRNA, microRNA, piRNA, lncRNA, and/or snoRNA). In some embodiments, the RNA assay assesses one or more genetic variants in the subject. In some embodiments, the RNA assay generates a profile comprising data on the subject's one or more genetic variants. Certain methods of isolating and assessing RNA are known to one skilled in the art. Certain exemplary methods include RNA-seq, qRT-PCR, two-tailed qRT-PCR, microarray, high-coverage capture sequencing (CaptureSeq), TaqMan miRNA assay, exome capture transcriptome sequencing, electrochemical detection, nanosensor, nanomechanical detection, and Single Molecule Array (Simoa). See, e.g., Stark et al. “RNA sequencing: the teenage years.” Nat Rev Genet 20, 631-656 (2019); Dufva “Introduction to microarray technology.” DNA Microarrays for Biomed Res 529, 1-22 (2009); Skrzypski “Quantitative reverse transcriptase real-time polymerase chain reaction (qRT-PCR) in translational oncology: lung cancer perspective.” 59:2, 147-154 (2008). In some embodiments, the RNA assay is RNA-seq.

In some embodiments, the test comprises an epigenetic assay. In some embodiments, the epigenetic assay generates a profile comprising data on epigenetic alterations. Certain exemplary data produced by an epigenetic assay include methylation state, chromatin compaction, histone modification, fragmentation patterns, topology, nucleosome positioning, and terminal overhang raggedness. Certain methods for assessing epigenetic modifications are known in the art. Certain exemplary methods include fractionation based on CpG methylation density using DNA capture with a methyl-CpG-binding domain protein (e.g., MBD2) followed by sequencing and bioinformatic analysis and DNA fragmentation pattern analysis (fragmentomics). See, e.g., Li and Zhou et al. “Methylation extends the reach of liquid biopsy in cancer detection.” Nat Rev Clin Oncol 17, 655-656 (2020); and Barefoot et al. “Detection of cell types contributing to cancer from circulating, cell-free methylated DNA.” Frontiers in Genetics 12, 1-14 (2021).

In some embodiments, an epigenetic assay comprises one or more of cell-free methylated DNA immunoprecipitation and high-throughput sequencing (cfMeDIP-seq), MeDIP-seq, methyl-CpG-binding domain sequencing (MBD-seq), methylated DNA capture sequencing (MethylCap-seq), hMe-Seal, Epi proColon, whole-genome bisulfite sequencing (WGBS), WGBS/CMS-IP-seq, bisulfite amplicon sequencing (BSAS), reduced representation bisulfite sequencing (RRBS), methylated CpG tandem amplification and sequencing (MCTA-seq), methylation array, methylation-specific PCR (MSP), Capture-seq, methylation-sensitive restriction enzyme sequencing (MRE-seq or MRSE-seq), MRSE-qPCR, Hpall-tiny fragment enrichment by ligation-mediated PCR (HELP), methyl-sensitive cut counting (MSCC), enzymatic methyl-sequencing (EM-seq), TET-assisted pyridine borane sequencing (TAPS), Tet-assisted bisulfite sequencing (TAB-seq), APOBEC-coupled epigenetic sequencing (ACE-seq), Tet-assisted 5-methylcytosine sequencing (TAmC-seq), oxidative bisulfite sequencing (oxBS-seq), nanopore-seq, single molecule real-time (SMRT)-seq, 450K array, targeted next-generation bisulfite sequencing (tNGBS), cell-free nucleosome occupancy and methylation sequencing (cfNOME-seq), PanSeer, assay for transposase-accessible chromatin with high-throughput sequencing (ATAC-seq), and single-cell ATAC-seq. In some embodiments, the epigenetic assay is a methylation assay. In some embodiments, the methylation assay comprises MBD-seq or MethyCap-Seq. In some embodiments, the methylation assay comprises a GRAIL Galleri test (also called the Galleri multicancer early detection (MCED) test). See, e.g., WO2021174072A1; WO2021202423A1; WO2021041840A1; WO2020163410A1; WO2021250677A1; Liu et al. “Sensitive and specific multi-cancer detection and localization using methylation signatures in cell-free DNA.” Ann Oncol 31:6, 745-759 (2020); and Klein et al. “Clinical validation of a targeted methylation-based multi-cancer early detection test using an independent validation set.” Ann Oncol 32:9, 1167-1177 (2021). In some embodiments, the methylation assay comprises a MethylMiner kit.

In some embodiments, the epigenetic assay generates a profile comprising data on fragmentomics. Certain methods for assessing DNA fragmentation patterns are known in the art. Exemplary methods include one or more of DNA evaluation of fragments for early interception (DELFI), large-scale co-fragmentation patterns (FREE-C), fragment coverage near transcription-start sites (TSS), cfDNA-accessibility score near the transcription factor-binding sites (TFBS), orientation-aware cfDNA fragmentation (OCF), windowed protection score (WPS), cfDNA-fragmentation hotspots, inference of DNA methylation from cfDNA-fragmentation patterns, the preferred-ended position of cfDNA, the end-motif frequency and motif-diversity score (MDS), jagged end, and patterns outside the chromosomes. See, e.g., Liu “At the dawn: cell-free DNA fragmentomics and gene regulation.” Br J Cancer 126, 379-390 (2022); and Lo et al. “Epigenetics, fragmentomics, and topology of cell-free DNA in liquid biopsies.” Science 372:6538, eaaw361 (2021).

In some embodiments, the test comprises a protein assay. In some embodiments, the protein assay generates a profile comprising data on peptide expression, protein expression, and/or the proteome. In some embodiments, the protein assay generates a profile comprising data on protein post-translational modifications. Certain methods for assessing individual peptides and proteins or the proteome are known to one skilled in the art. Exemplary methods include one or more of an enzyme-linked immunosorbent assay (ELISA), chemiluminescence immunoassay (CLIA), immunohistochemistry (IHC), liquid-bead immunoassay, immunoblotting, antibody array, antigen array, reverse phase protein array (RPPA), proximity extension assay (PEA), bead-based array, liquid chromatography-mass spectrometry (LC-MS/MS), Multi-Dimensional Protein Identification Technology (MudPIT), surface-enhanced laser desorption/ionization (SELDI)-MS, slow off-rate modified aptamers (SOMA) scan assay, AptoDetect-Lung, and OVERA. See, e.g., Ding et al. “Proteomics technologies for cancer liquid biopsies.” Mol Cancer 21:53, (2022).

In some embodiments, the test comprises a metabolic assay. In some embodiments, the metabolic assay generates a profile comprising metabolic and/or metabolomic data. Certain methods for assaying the metabolome use nuclear magnetic resonance spectroscopy (NMR-spec) or are mass spectrometry-based. See, e.g., McCartney et al. “Metabolomics in breast cancer: a decade in review.” Cancer Treat Rev 67, 88-96 (2018); and Spratlin et al. “Clinical applications of metabolomics in oncology: a review.” Clin Cancer Res 15:2, 431-440 (2009). In some embodiments, the metabolic assay comprises one or more of nuclear magnetic resonance spectroscopy (NMR-spec), gas chromatography-mass spectrometry (GC-MS), LC-MS, and Fourier transform ion cyclotron resonance (Fourier transform/MS).

In some embodiments, the test comprises a microbiome assay. In some embodiments, the microbiome assay generates a microbial profile. In some embodiments, a microbiome can be profiled by sequencing methods known in the art. See, e.g., Adlung et al. “Microbiome genomics for cancer prediction.” Nat Cancer 1, 379-381 (2020); Dzutsev and Trinchieri “Microbial DNA signature in plasma enables cancer diagnosis.” Nat Rev Clin Oncol 17, 453-454 (2020); and Riquelme et al. “Tumor microbiome diversity and composition influence pancreatic cancer outcomes.” Cell 178:4, 795-806 (2019). In some embodiments, the microbiome assay comprises one or more of RNA-seq, WGS, and 16S ribosomal RNA sequencing.

In some embodiments, the test comprises an assay that assesses circulating tumor cells (CTCs). In some embodiments, circulating tumor cells are enriched from the biological sample using size exclusion or protein expression markers using methods known in the art. See, e.g., Zhang et al. “Integrative diagnosis of cancer by combining CTCs and associated peripheral blood cells in liquid biopsy.” Clin Transl Oncol 21:7, 828-835 (2019) and Hofman et al. “Liquid biopsy in the era of immune-oncology: is it ready for prime-time use for cancer patients?” Ann Oncol 30:9, 1448-1459 (2019). Certain methods for detecting circulating tumor cells include CellSearch, CellSieve, EPIC CTC Platform, Vortex, ISET, negative depletion, magnetic separation, Maintrac, RT-qPCR, microfluidic device, and GO chip. In some embodiments, circulating tumor cells express one or more of epithelial cell adhesion molecule (EpCAM), programmed death receptor ligand-1 (PD-L1), cytotoxic T-lymphocyte-associated protein 4 (CTLA4), cytokeratin (CK), and RAD50. In some embodiments, circulating tumor cells are identified by absence of CD45. Once isolated, in some embodiments, circulating tumor cells are assayed for nucleic acid, epigenetic, peptide, protein, and/or metabolic biomarkers.

In some embodiments, the test comprises an assay that assesses immune cell populations. In some embodiments, the immune cell assay generates an immune cell profile. Certain methods for assessing immune cells from liquid biopsies are known in the art. Certain exemplary methods include one or more of qRT-PCR, RNA-seq, single-cell RNA-seq, NanoString's nCounter, flow cytometry, phosphoflow, cytometry by time-of-flight (CyTOF), microengraving, and barcoded microchip assay. See, e.g., Lyons et al. “Immune cell profiling in cancer: molecular approaches to cell-specific identification.” Npj Precision Onc 1:26, 1-8 (2017).

In some embodiments, the test comprises an assay that assesses extracellular vesicles. In some embodiments, the extracellular vesicles are tumor-derived exosomes. Tumor-derived exosomes are known to circulate in the bloodstream and can be isolated by certain methods known to one skilled in the art. See, e.g., WO2019068269A1; Yoshioka et al. “Ultra-sensitive liquid biopsy of circulating extracellular vesicles using ExoScreen.” Nat Commun 5, 3591 (2014); Logozzi et al. “Exosomes: a source for new and old biomarkers in cancer.” Cancers 12:9, 2566 (2020); and Makler and Asghar “Exosomal biomarkers for cancer diagnosis and patient monitoring.” Expert Rev Mol Diagn 20:4, 387-400 (2020). In some embodiments, the exosomes are enriched by ultrafiltration, ultracentrifugation, sucrose gradient ultracentrifugation, size-exclusion chromatography, immunocapture, immunoaffinity, immunoprecipitation, polymeric precipitation, extracellular vesicle array, ExoScreen, immunocapture-based enzyme-linked immunosorbent assay (IC-ELISA), nanosensor, or microfluidic-based assays.

In some embodiments, the test assesses both epigenetic alterations (e.g., methylation status) and nucleic acids, wherein the nucleic acid test comprises both DNA and RNA assays. See, e.g., WO2017181146A1. In some embodiments, the test comprises a Guardant Reveal test. In some embodiments, the test comprises a Shield test. See, e.g., Kim et al. “Combined genomic and epigenomic assessment of cell-free circulating tumour DNA (ctDNA) improves assay sensitivity in early stage colorectal cancer (CRC).” Cancer Res. 79(suppl 13), 916 (2019); Liles et al. “Uptake of a colorectal cancer screening blood test is higher than of a fecal test offered in clinic: a randomized trial.” Cancer Treat Res Comm. 10, 27-31 (2017); Westesson et al. “Integrated genomic and epigenomic cell-free DNA (cfDNA) analysis for the detection of early-stage colorectal cancer.” Cancer Res. 80(suppl 16), 2316 (2020); Mack et al. “Residual circulating tumor DNA (ctDNA) after two months of therapy to predict progression-free and overall survival in patients treated on 51403 with afatinib+/−cetuximab.” J Clin Oncol 38:15_suppl, 9532-9532 (2020).

In some embodiments, the test assesses both nucleic acids and proteins. In some embodiments, the test comprises a CancerSEEK test. See, e.g., WO2020150656A1; Cohen et al. “Detection and localization of surgically resectable cancer with a multi-analyte blood test.” Science 359:6378, 926-930 (2018). In some embodiments, the test is a multiomics assay. In some embodiments, the multiomics assay comprises the Freenome multiomics platform. See, e.g., WO2021202351A1; WO2021222220A2; UIz et al. “Inference of transcription factor binding from cell-free DNA enables tumor subtype prediction and early detection.” Nat Commun 10:4666 (2019); Wan et al. “Machine learning enables detection of early-stage colorectal cancer by whole-genome sequencing of plasma cell-free DNA.” BMC Cancer 19:832 (2019); and Ignjatovic et al. “Mass spectrometry-based plasma proteomics: considerations from sample collection to achieving translational data.” J. Proteome Res 18:12, 4085-4097 (2019). In some embodiments, the test comprises LUNAR-2. See, e.g., Kim et al. “Combined genomic and epigenomic assessment of cell-free circulating tumor DNA (ctDNA) improves assay sensitivity in early-stage colorectal cancer (CRC).” Cancer Res 79: 13 supplement, Abstract 916 (2019).

In some embodiments, the assay or assays is available as a kit. In some embodiments, the assay or assays is available as a kit that is approved for clinical use.

### First and Second Tests

In some embodiments, a first liquid biopsy test is performed, wherein the profile generated by the first test determines whether to perform a second test. In some embodiments, the second test is a second liquid biopsy test.

In some embodiments, the first test detects a cancer, and the second test is performed to identify one or more druggable targets (e.g., to select one or more suitable treatments) for treating the cancer. In some embodiments, the subject is treated with the one or more drugs selected according to the second test.

In some embodiments, the second test assesses the presence or absence of one or more genetic variants that identify one or more druggable targets. In some embodiments, the second test identifies an effective dosage for a drug that targets the one or more druggable targets. In some embodiments, the second test eliminates one or more possible treatments as unsuitable for a subject, e.g., by identifying a high likelihood of resistance to a given drug, e.g., based on nucleic acid data.

In some embodiments, the first test does not detect a cancer and the second test is not performed. Without being limited by theory, such embodiments offer a more cost-effective and targeted alternative to conventional methods that combine cancer screening with comprehensive and expensive testing, including for subjects who do not have cancer and thus do not require such testing.

In some embodiments, a first liquid biopsy test and a second test are performed, wherein a profile or result from the first liquid biopsy test determines the selection of the second test. In some embodiments, the second test is a second liquid biopsy test.

In some embodiments, the profile from the first test narrows a panel of possible second tests to a specific second test. In some embodiments, the profile from the first test narrows a panel of possible druggable targets in a subject to a targeted panel of possible druggable targets, e.g., to be assessed by a second test. In some embodiments, the profile from the first test narrows a panel of possible genetic variants in a subject to a targeted panel of possible genetic variants, e.g., to be assessed by a second test. For example, in some embodiments, the profile from the first test narrows a panel of possible genetic variants to be assessed by the second test from, e.g., at least 40, 50, 60, 70, 80, 90, 100, 150, 200, 250, or 300 possible variants, to ten or fewer possible variants, preferably five or fewer (e.g., 5, 4, 3, 2, or 1) possible variants. Without being limited by theory, such embodiments offer a more cost-effective and targeted alternative to conventional methods that run comprehensive and expensive testing to determine or eliminate treatment options. Rather, embodiments described herein help focus testing to the most likely candidates, thus facilitating a more efficient and less expensive path to treatment as compared to conventional methods.

In some embodiments, the first test produces more than one profile, wherein each profile is predictive of one or more druggable targets in the subject. In some embodiments, the first test can produce 2, 3, 4, 5, 6, 7, 8, 9, 10, or more profiles. In some embodiments, the one or more druggable targets predicted by each profile may overlap but is not identical. In some embodiments, the one or more druggable targets predicted by each profile is unique for each profile. In some embodiments, a second test (optionally a second liquid biopsy test) is selected according to the profile generated by the first test. In some embodiments, the second test is selected from a panel of possible second tests, wherein each possible second test assesses the presence or absence of one or more druggable targets. In some embodiments, the one or more druggable targets assessed by each possible second test may overlap but is not identical. In some embodiments, the one or more druggable targets assessed by each possible second test is unique. Without being limited by theory, such embodiments offer a more cost-effective and targeted alternative to conventional methods that run comprehensive and expensive testing to determine or eliminate treatment options. Rather, embodiments described herein help focus testing to the most likely candidates, thus facilitating a more efficient and less expensive path to treatment as compared to conventional methods.

In some embodiments, the first test produces a first or second profile, wherein the first profile is predictive of a first panel of one or more druggable targets in the subject and the second profile is predictive of a second panel of one or more druggable targets in the subject. In some embodiments, the first profile generated by the first test predicts one or more druggable targets in a first pathway, and the second profile generated by the first test predicts one or more druggable targets in a second pathway. In some embodiments, the first test generates the first profile, and the second test assesses the presence or absence of the one or more druggable targets of the first pathway. In some embodiments, the first test generates the second profile, and the second test assesses the presence or absence of the one or more druggable targets of the second pathway. In some embodiments, the first and second pathways have one or more overlapping druggable targets, but not an identical panel of possible druggable targets. In some embodiments, the first and second pathways have no overlapping possible druggable targets. In some embodiments, at least one of the one or more druggable targets is a genetic variant. In some embodiments, at least one of the one or more druggable targets is a peptide or protein.

In some embodiments, one or more calling criteria for the second test is adjusted based on a profile generated by the first test. In some embodiments, the profile comprises data from multiple assays (i.e., any combination of assays described herein). For example, in some embodiments, a profile generated by the first test comprises nucleic acid data (e.g., data predicting the likelihood of one or more genetic variants), and data from another assay (e.g., methylation state data, microbiome data, protein sequence or post-translational modification data, fragmentation patterns, metabolomics data), and the conventional detectable threshold for predicting a genetic variant is lowered in view of the data from the other assay. In other words, in some embodiments, the data of a profile generated by the first test are weighted to select likely genetic variants or likely druggable targets in a subject, and thus to select a second test from a panel of possible second tests. Without being limited by theory, such embodiments improve upon conventional methods by applying adjusted (e.g., lower) thresholds or criteria that facilitate accurate diagnosis and treatment of subjects.

In some embodiments, the first test generates one or more profiles that identify a cancer in a subject. In some embodiments, the first test generates one or more profiles that indicates the absence of cancer in a subject. In some embodiments, the one or more profiles generated by the first test predict the tissue-of-origin of a cancer in the subject. In some embodiments, the one or more profiles generated by the first test identify the tissue-of-origin of a cancer in the subject. In some embodiments, the one or more profiles generated by the first test predict one or more druggable targets in the subject. In some embodiments, the one or more profiles generated by the first test guide the selection of an appropriate second test from a panel of possible second tests for confirming (e.g., detecting) or eliminating one or more druggable targets predicted by the one or more profiles generated by the first test.

In some embodiments, the second test generates one or more profiles that identify one or more druggable targets in the subject. In some embodiments, the second test generates one or more profiles that assess the presence or absence of one or more druggable targets predicted by the first test. In some embodiments, the second test generates one or more profiles that confirm the presence of one or more druggable targets predicted by the first test. In some embodiments, the one or more profiles generated by the second test guide the selection of an appropriate treatment for a cancer. In some embodiments, the one or more profiles generated by the second test identify one or more appropriate and/or unsuitable treatments for a cancer. In some embodiments, an appropriate treatment is one to which the cancer is likely to be susceptible. In some embodiments, an appropriate treatment is one to which the cancer is unlikely to be resistant. In some embodiments, the second test identifies an effective dosage for the appropriate treatment. In some embodiments, the one or more profiles generated by the second test identify that a cancer is resistant or is likely to be resistant to one or more potential treatments, thus identifying that those one or more potential treatments are unsuitable.

In some embodiments, the first test comprises a DNA assay. In some embodiments, the first test comprises an RNA assay. In some embodiments, the first test comprises an epigenetic assay. In some embodiments, the first test comprises a methylation assay. In some embodiments, the first test comprises a fragmentomics assay. In some embodiments, the first test comprises a protein assay. In some embodiments, the first test comprises a metabolic assay. In some embodiments, the first test comprises a microbiome assay. In some embodiments, the first test assesses circulating tumor cells. In some embodiments, the first test assesses immune cells. In some embodiments, the first test assesses extracellular vesicles. In some embodiments, the first test comprises a DNA assay, a methylation assay, a fragmentomics assay, and a protein assay.

In some embodiments, the second test comprises a DNA assay. In some embodiments, the second test comprises an RNA assay. In some embodiments, the second test comprises an epigenetic assay. In some embodiments, the second test comprises a methylation assay. In some embodiments, the second test comprises a fragmentomics assay. In some embodiments, the second test comprises a protein assay. In some embodiments, the second test comprises a metabolic assay. In some embodiments, the second test comprises a microbiome assay. In some embodiments, the second test assesses circulating tumor cells. In some embodiments, the second test assesses immune cells. In some embodiments, the second test assesses extracellular vesicles. In some embodiments, the second test comprises a DNA assay, a methylation assay, a fragmentomics assay, and a protein assay.

In some embodiments, the first test identifies the presence or absence of a cancer in a subject, the first test comprises an epigenetic assay, and the second test comprises a nucleic acid assay (e.g., a DNA assay and/or an RNA assay). In some embodiments, the epigenetic assay generates both a methylation profile and a fragmentomics profile.

In some embodiments, the first test identifies the presence or absence of a cancer in a subject, the first test comprises an epigenetic assay, and the second test comprises a DNA assay and a protein assay. In some embodiments, the epigenetic assay generates both a methylation profile and a fragmentomics profile.

In some embodiments, the first test identifies the presence or absence of a cancer in a subject, the first test comprises an epigenetic assay, and the second test comprises an RNA assay and a protein assay. In some embodiments, the epigenetic assay generates both a methylation profile and a fragmentomics profile.

In some embodiments, the first test identifies the presence or absence of a cancer in a subject, the first test comprises an epigenetic assay, and the second test comprises a DNA assay, an RNA assay, and a protein assay. In some embodiments, the epigenetic assay generates both a methylation profile and a fragmentomics profile.

In some embodiments, the first test identifies the presence or absence of a cancer in a subject, the first test comprises an epigenetic assay, and the second test comprises a methylation assay, a fragmentomics assay, a nucleic acid assay (e.g., a DNA assay and/or an RNA assay), and a protein assay.

In some embodiments, the first test predicts one or more druggable targets, the first test comprises an epigenetic assay, and the second test comprises a DNA assay. In some embodiments, the epigenetic assay generates both a methylation profile and a fragmentomics profile.

In some embodiments, the first test predicts one or more druggable targets, the first test comprises an epigenetic assay, and the second test comprises a protein assay. In some embodiments, the epigenetic assay generates both a methylation profile and a fragmentomics profile.

In some embodiments, the first test predicts one or more druggable targets, the first test comprises a DNA assay and an epigenetic assay, and the second test comprises a protein assay. In some embodiments, the epigenetic assay generates both a methylation profile and a fragmentomics profile.

In some embodiments, the first test predicts one or more druggable targets, the first test comprises a DNA assay and an epigenetic assay, and the second test comprises a nucleic acid assay (e.g., a DNA assay and/or an RNA assay). In some embodiments, the epigenetic assay generates both a methylation profile and a fragmentomics profile.

In some embodiments, the first test predicts one or more druggable targets, and the first test comprises a methylation assay and a fragmentomics assay. In some embodiments, the first test predicts one or more druggable targets, the first test comprises a methylation assay and a fragmentomics assay, and the second test comprises a nucleic acid assay. In some embodiments, the first test predicts one or more druggable targets, the first test comprises a methylation assay and a fragmentomics assay, and the second test comprises a protein assay. In some embodiments, the first test predicts one or more druggable targets, the first test comprises a methylation assay and a fragmentomics assay, and the second test comprises a nucleic acid assay and a protein assay.

In some embodiments, the first test predicts one or more druggable targets, and the first test comprises a methylation assay, a fragmentomics assay, and a nucleic acid assay. In some embodiments, the first test predicts one or more druggable targets, the first test comprises a methylation assay, a fragmentomics assay, and a nucleic acid assay, and the second test comprises a further nucleic acid assay. In some embodiments, the first test predicts one or more druggable targets, the first test comprises a methylation assay, a fragmentomics assay, and a nucleic acid assay, and the second test comprises a protein assay.

In some embodiments, the first test predicts one or more druggable targets, and the first test comprises a methylation assay, a fragmentomics assay, a nucleic acid assay, and a protein assay. In some embodiments, the first test predicts one or more druggable targets, the first test comprises a methylation assay, a fragmentomics assay, a nucleic acid assay, and a protein assay, and the second test comprises a further nucleic acid assay. In some embodiments, the first test predicts one or more druggable targets, the first test comprises a methylation assay, a fragmentomics assay, a nucleic acid assay, and a protein assay, and the second test comprises a further protein assay.

### Sets

In some embodiments, a first test and a second test are provided in a set. In some embodiments, both the first and second tests of the set are performed. In some embodiments, only the first test of the set is performed. In some embodiments, the first test indicates that a subject does not have a cancer, and the second test of the set (e.g., to identify one or more druggable targets for treating cancer) is not performed.

In some embodiments, the second test of the set assesses the presence or absence of one or more genetic variants that identify one or more druggable targets. In some embodiments, the second test of the set confirms the presence of one or more genetic variants that identify one or more druggable targets as predicted by the first test. In some embodiments, the second test of the set identifies an effective dosage for a drug that targets the one or more druggable targets.

In some embodiments, the profile from the first test of the set narrows a panel of possible second tests to a specific second test. In some embodiments, the profile from the first test of the set narrows a panel of possible druggable targets in a subject to a targeted panel of possible druggable targets. In some embodiments, the profile from the first test of the set narrows a panel of possible genetic variants in a subject to a targeted panel of possible genetic variants.

In some embodiments, the first test of the set produces more than one profile, wherein each profile is predictive of one or more druggable targets in the subject. In some embodiments, the first test of the set can produce 2, 3, 4, 5, 6, 7, 8, 9, 10, or more profiles. In some embodiments, a second test of the set (optionally a second liquid biopsy test) is selected according to the profile generated by the first test of the set. In some embodiments, the second test of the set is selected from a panel of possible second tests, wherein each second test assesses the presence or absence of one or more druggable targets.

In some embodiments, the first test of the set generates one or more profiles that identify a cancer in a subject. In some embodiments, the first test of the set generates one or more profiles that indicates the absence of cancer in a subject. In some embodiments, the one or more profiles generated by the first test of the set predict the tissue-of-origin of a cancer in the subject. In some embodiments, the one or more profiles generated by the first test of the set identify the tissue-of-origin of a cancer in the subject. In some embodiments, the one or more profiles generated by the first test of the set predict one or more druggable targets in the subject. In some embodiments, the one or more profiles generated by the first test of the set guide the selection of an appropriate second test from a panel of possible second tests for confirming (e.g., detecting) or eliminating one or more druggable targets predicted by the one or more profiles generated by the first test.

In some embodiments, one or more calling criteria for the second test of the set is adjusted based on a weighted profile generated by the first test of the set. In some embodiments, the profile comprises data from multiple assays (i.e., any combination of assays described herein).

In some embodiments, the second test of the set generates one or more profiles that identify one or more druggable targets in the subject. In some embodiments, the second test of the set generates one or more profiles that assess the presence or absence of one or more druggable targets predicted by the first test. In some embodiments, the one or more profiles generated by the second test of the set guide the selection of an appropriate treatment for a cancer. In some embodiments, the one or more profiles generated by second test of the set identify one or more appropriate and/or unsuitable treatments for a cancer. In some embodiments, an appropriate treatment is one to which the cancer is likely to be susceptible. In some embodiments, an appropriate treatment is one to which the cancer is unlikely to be resistant. In some embodiments, the second test of the set identifies an effective dosage for the appropriate treatment. In some embodiments, the one or more profiles generated by the second test of the set identify that a cancer is resistant or is likely to be resistant to one or more potential treatments, thus identifying that those one or more potential treatments are unsuitable.

In some embodiments, the first test of the set comprises a DNA assay. In some embodiments, the first test of the set comprises an RNA assay. In some embodiments, the first test of the set comprises an epigenetic assay. In some embodiments, the first test of the set comprises a methylation assay. In some embodiments, the first test of the set comprises a fragmentomics assay. In some embodiments, the first test of the set comprises a protein assay. In some embodiments, the first test of the set comprises a metabolic assay. In some embodiments, the first test of the set comprises a microbiome assay. In some embodiments, the first test of the set assesses circulating tumor cells. In some embodiments, the first test of the set assesses immune cells. In some embodiments, the first test of the set assesses extracellular vesicles. In some embodiments, the first test of the set comprises a DNA assay, a methylation assay, a fragmentomics assay, and a protein assay.

In some embodiments, the second test of the set comprises a DNA assay. In some embodiments, the second test of the set comprises an RNA assay. In some embodiments, the second test of the set comprises an epigenetic assay. In some embodiments, the second test of the set comprises a methylation assay. In some embodiments, the second test of the set comprises a fragmentomics assay. In some embodiments, the second test of the set comprises a protein assay. In some embodiments, the second test of the set comprises a metabolic assay. In some embodiments, the second test of the set comprises a microbiome assay. In some embodiments, the second test of the set assesses circulating tumor cells. In some embodiments, the second test of the set assesses immune cells. In some embodiments, the second test of the set assesses extracellular vesicles. In some embodiments, the second test of the set comprises a DNA assay, a methylation assay, a fragmentomics assay, and a protein assay.

In some embodiments, the first test of the set identifies the presence or absence of a cancer in a subject, the first test of the set comprises an epigenetic assay, and the second test of the set comprises a nucleic acid assay (e.g., a DNA assay and/or an RNA assay). In some embodiments, the epigenetic assay generates both a methylation profile and a fragmentomics profile.

In some embodiments, the first test of the set identifies the presence or absence of a cancer in a subject, the first test of the set comprises an epigenetic assay, and the second test of the set comprises a DNA assay and a protein assay. In some embodiments, the epigenetic assay generates both a methylation profile and a fragmentomics profile.

In some embodiments, the first test of the set identifies the presence or absence of a cancer in a subject, the first test of the set comprises an epigenetic assay, and the second test of the set comprises an RNA assay and a protein assay. In some embodiments, the epigenetic assay generates both a methylation profile and a fragmentomics profile.

In some embodiments, the first test of the set identifies the presence or absence of a cancer in a subject, the first test of the set comprises an epigenetic assay, and the second test of the set comprises a DNA assay, an RNA assay, and a protein assay. In some embodiments, the epigenetic assay generates both a methylation profile and a fragmentomics profile.

In some embodiments, the first test of the set identifies the presence or absence of a cancer in a subject, the first test of the set comprises an epigenetic assay, and the second test of the set comprises a methylation assay, a fragmentomics assay, a nucleic acid assay (e.g., a DNA assay and/or an RNA assay), and a protein assay.

In some embodiments, the first test of the set predicts one or more druggable targets, the first test of the set comprises an epigenetic assay, and the second test of the set comprises a DNA assay. In some embodiments, the epigenetic assay generates both a methylation profile and a fragmentomics profile.

In some embodiments, the first test of the set predicts one or more druggable targets, the first test of the set comprises an epigenetic assay, and the second test of the set comprises a protein assay. In some embodiments, the epigenetic assay generates both a methylation profile and a fragmentomics profile.

In some embodiments, the first test of the set predicts one or more druggable targets, the first test of the set comprises a DNA assay and an epigenetic assay, and the second test of the set comprises a protein assay. In some embodiments, the epigenetic assay generates both a methylation profile and a fragmentomics profile.

In some embodiments, the first test of the set predicts one or more druggable targets, the first test of the set comprises a DNA assay and an epigenetic assay, and the second test of the set comprises a nucleic acid assay (e.g., a DNA assay and/or an RNA assay). In some embodiments, the epigenetic assay generates both a methylation profile and a fragmentomics profile.

In some embodiments, the first test of the set predicts one or more druggable targets, and the first test of the set comprises a methylation assay and a fragmentomics assay. In some embodiments, the first test of the set predicts one or more druggable targets, the first test of the set comprises a methylation assay and a fragmentomics assay, and the second test of the set comprises a nucleic acid assay. In some embodiments, the first test of the set predicts one or more druggable targets, the first test of the set comprises a methylation assay and a fragmentomics assay, and the second test of the set comprises a protein assay. In some embodiments, the first test of the set predicts one or more druggable targets, the first test of the set comprises a methylation assay and a fragmentomics assay, and the second test comprises a nucleic acid assay and a protein assay.

In some embodiments, the first test of the set predicts one or more druggable targets, and the first test of the set comprises a methylation assay, a fragmentomics assay, and a nucleic acid assay. In some embodiments, the first test of the set predicts one or more druggable targets, the first test of the set comprises a methylation assay, a fragmentomics assay, and a nucleic acid assay, and the second test of the set comprises a further nucleic acid assay. In some embodiments, the first test of the set predicts one or more druggable targets, the first test of the set comprises a methylation assay, a fragmentomics assay, and a nucleic acid assay, and the second test of the set comprises a protein assay.

In some embodiments, the first test of the set predicts one or more druggable targets, and the first test of the set comprises a methylation assay, a fragmentomics assay, a nucleic acid assay, and a protein assay. In some embodiments, the first test of the set predicts one or more druggable targets, the first test of the set comprises a methylation assay, a fragmentomics assay, a nucleic acid assay, and a protein assay, and the second test of the set comprises a further nucleic acid assay. In some embodiments, the first test of the set predicts one or more druggable targets, the first test of the set comprises a methylation assay, a fragmentomics assay, a nucleic acid assay, and a protein assay, and the second test of the set comprises a further protein assay.

### Biomarkers and Genetic Variants

The first test of the methods, kits, and sets described herein may predict or identify biomarkers in a subject, e.g., biomarkers predictive of treatment efficacy or resistance. The first profile of the methods, kits, and sets described herein may predict or identify biomarkers in a subject, e.g., biomarkers predictive of treatment efficacy or resistance. The second test of the methods, kits, and sets described herein may identify or assess the presence or absence of one or more biomarkers in a subject, e.g., biomarkers predictive of treatment efficacy or resistance. Biomarkers may include, but are not limited to, druggable targets. Further, biomarkers may include, but are not limited to, a nucleic acid (e.g., a genetic variant in the subject or the subject's microbiome), an epigenetic marker, a peptide, a protein, a lipid, or an immune cell profile, e.g., as described further herein.

As used herein, “genetic variant” means any alteration in a gene or gene product (e.g., RNA, peptide, and/or protein) and refers to, e.g., the presence of a mutation or mutations within the gene or gene product, an alteration in copy number of the gene or gene product, or a translocation of the gene or gene product. The genetic variant may affect the integrity, sequence, structure, amount, or activity of the gene or gene product as compared to the wild-type gene. Certain exemplary genetic variants include those relating to the nucleic acid sequence of all or a portion of the genome (e.g., nucleotide polymorphism, indel, sequence rearrangement, mutational frequency, and/or chromosomal translocation), the copy number of one or more particular nucleotide sequences within the genome (e.g., copy number, single chromosome or entire genome ploidy, and/or allele frequency fractions), and the expression profile of the organism's genome (e.g., gene expression levels, isotype expression levels, and/or gene expression ratios). In some embodiments, the genetic variant comprises a gene fusion. In some embodiments, the genetic variant is a druggable target. In some embodiments, the genetic variant is a biomarker for a druggable target.

In some embodiments, the DNA profile comprises a genetic variant that indicates a biomarker. Certain cancer-associated biomarkers are known to one skilled in the art. Certain exemplary biomarkers include ALK, AKT1, APC, AR (androgen receptor), ASXL1, ATM, BRAF, BRCA1, BRACA2, c-Kit, c-MET, CDK4, CDK12, CDKN2A, CTNNB1, DNMT3A, EGFR, ERa, ERBB2, ESR1, FBXW7, FGFR1, FGFR2, FGFR3, GNAS, HER2, HRAS, IDH1, IDH2, JAK2, KRAS (including variants G12C, G12D, and G12R), MED1, MEK, MET, MLH1, MSH2, MSH6, NTRK, NRAS, PDGFR, PIK3CA, PMS2, POLE, POLD, PPP2R1A, PTEN, Rb1, ROS1, RET, ROS1, TET2, TP53, VEGF, and VEGFR. In some embodiments, the biomarker comprises microsatellite instability (MSI) status.

In some embodiments, the DNA profile comprises a genetic variant that indicates a signaling pathway associated with tumorigenesis, e.g., the cell cycle, Hippo, Myc, Notch, oxidative stress response/Nrf2, RAS/MAPK, Akt/PI3K/mTORC1, TGFβ, p53, and/or β-catenin/Wnt signaling pathways. See, e.g., Sanchez-Vega et al. “Oncogenic signaling pathways in The Cancer Genome Atlas.” Cell 173:2, 321-337.e10 (2018).

In some embodiments, the RNA profile comprises RNA biomarkers. In some embodiments, the RNA biomarker comprises an RNA molecule. Certain exemplary RNA molecules include mRNA, long noncoding RNA (lncRNA), microRNA, small nucleolar RNA (snoRNA), circular RNA (circRNA), and Piwi-interacting RNA (piRNA). In some embodiments, an mRNA biomarker is the translated product of any DNA genetic variant biomarker described herein. Certain noncoding RNA biomarkers are known in the art. Certain exemplary RNA biomarkers include ACO21218.2, AFAP1-AS1, ANRIL, BALR-1, BALR-2, BALR-6, BANCR, CCAT-2, CRNDE.h, FALEC, FAM83H-AS-1, GAPLINC, GAS-5, H19, HOTAIR, HOX-AS-S, HYMA-1, LIMT, LINC00310, LINC00858, LINC00958, LincRNA-P21, Linc-ROR, LINK-00477, lncRNA-LRB1, Lnc-PCDH9-13:1, lncRNA-P21, LOC_152578, LOC100506688, LOC149086, MALAT-1, MIAT, NEAT-1, NR_026817, NR_029373, NR_034119, OTX2-AS1, P34822, PCA3, PCAT18, PVT-1, RP11-138J23.1, RP11-160H22.5, RP11-317J10.2, RP11-43505.2, RP11-445H22.4, SNHG-6, SPRY4-IT1, TUBA-4B, TUG-1, UCA-1, XLOC_000303, XLOC_006844, XLOC-014172, XLOC-109948, ZFAS1, piR-651, piR-823, miR-10b, miR-17-5p, miR-18a, miR-18b-5p, miR-19a-3p, miR-20a-5p, miR-21, miR-29a, miR-34a, miR-93-5p, miR-98-5p, miR-101-3p, miR-103-3p, miR-106a, miR-107, miR-122, miR-125, miR-125b, miR-126-3p, miR-126-5p, miR-130a-3p, miR-141, miR-144-3p, miR-144-5p, miR-145, miR-155, miR-190a-5p, miR-192, miR-193b, miR-194, miR-200a, miR-200b, miR-210, miR-221, miR-222, miR-224, miR-301, miR-301a-3p, miR-301b-3p, miR-454-3p, miR-664b-5p, miR-1246, miR-1275, miR-4485-5p, miR-5793, miR-6749-5p, miR-200c, miR-375, let-7f-5p, let-7d-5p, SNORA25, SNORD33, SNORD66, SNORA74A, and SNORD76. See, e.g., WO2018055093A1; Xi et al. “RNA biomarkers: frontier of precision medicine for cancer.” Noncoding RNA 3:1, 9 (2017); and Sarfi et al. “Long noncoding RNAs: biomarker-based assessment.” J Cell Phys 234:10, 16971-16986 (2019).

In some embodiments, the epigenetic profile comprises information regarding specific epigenetic alterations, e.g., methylation, 5-methylcytosine (5mC), hydroxymethylation, 5-hydroxymethylcytosine, N6-methyladenine, chromatin accessibility, accessibility of transcription factor binding sites, histone occupancy, terminal overhang raggedness, and/or DNA fragment length. Methylation status and other epigenetic modifications are known to be correlated with the presence of some disease conditions such as cancer, and specific patterns of methylation have been determined to be associated with particular cancer conditions. See, e.g., WO2022002423A1; Jones “DNA methylation and cancer.” Oncogene 21:35, 5358-5360 (2002); and Paska and Hudler, “Aberrant methylation patters in cancer: a clinical view.” Biochemia Med 25:2, 161-176 (2015). Methylation patterns can also be observed in cell-free DNA. See Warton and Samimi, “Methylation of cell-free circulating DNA in the diagnosis of cancer.” Front Mol Biosci, 2:13 (2015).

In some embodiments, the epigenetic profile comprises methylation data that indicates a cancer biomarker. In some embodiments, the methylated biomarker may comprise a gene or genes that are hypermethylated. Certain genes known in the art are silenced via promoter hypermethylation in cancer. Certain exemplary hypermethylated genes include Rb, p16INK4a, BRCA1, VHL, CDH1, MLH1, IGSF4, HOXA, PCDH, SEMA3F, SEPT9, WIFI, DACT2, SOSTDC1, BRMS1, SLC6A1, F13A1, BARHL1, CSM D2, and chromosome region 2q14.2. See, e.g., WO2022013880A1. In some embodiments, the methylated biomarker may comprise a gene or genes that are hypomethylated. Certain exemplary genes known in the art that are hypomethylated include Jagged1 genes, Notch genes, hTERT, or Iroquois homeobox 1 (IRX1). In some embodiments, the methylated biomarker may comprise a non-coding RNA molecule, e.g., a microRNA. Certain non-coding RNAs that have differential epigenetic regulation compared to wild-type epigenetic regulation are a biomarker for cancer diagnosis. See, e.g., Wang et al. “Mutual regulation of microRNAs and DNA methylation in human cancers.” Epigenetics 12:3, 187-197 (2017). Certain exemplary hypermethylated microRNA biomarkers include miR-137, miR-124-2, miR-124-3, miR-9-3, miR-203a, miR-148a, miR-34b/c, miR-34a, miR-203, miR-124, and miR-212. In some embodiments, microRNA biomarkers comprise hypomethylated genes, e.g., miR135b.

In some embodiments, the peptide, protein, or proteomics profile comprises a biomarker. In some embodiments, the biomarker comprises a peptide or protein biomarker. In some embodiments, the peptide or protein biomarker is a cancer-associated peptide or protein biomarker. Certain exemplary peptide or protein biomarkers include AFP (alpha-fetoprotein), breast cancer resistance protein (BCRP), carbohydrate antigen 15-3 (CA 15-3), CA 19-9, CA 27-29, CA-125, carbonic anhydrase IX (CA IX), carcinoembryonic antigen (CEA), developmental endothelial locus-1 (Del-1), fibronectin, gastrokine 1 (GKN1), glycoprotein leucine-rich a-2 glycoprotein 1 (LRG1), glypican-1 (GPC1), soluble HER2 (sHER2), human chorionic gonadotropin β-subunit (hCG-13), human growth factor (HGF), interleukin-8 (IL-8), leucine-rich alpha-2-glycoprotein 1 (LRG1), leptin, melanoma inhibitory activity (MIA), mucin 16, NY-ESO-1, osteopontin (OPN), soluble PD-L1, Prolactin, prostate-specific antigen (PSA), S100B, survivin, thrombospondin-2 (THBS2), tissue polypeptide antigen (TPA), tissue polypeptide-specific antigen (TPS), apolipoprotein CI, apolipoprotein (a), neural cell adhesion molecule LI-like protein, carbonic anhydrase 1, Olfactomedin-4, neudesin, desmoplakin, and tissue inhibitor of metalloproteinase 1 (TIMP1).

In some embodiments, the peptide or protein biomarker comprises a post-translational modification to a peptide or protein. In some embodiments, the protein post-translational modification comprises phosphorylation, glycosylation, lipidation, nitrosylation, ubiquitination, methylation, hydroxylation, and/or acetylation.

In some embodiments, the metabolic or metabolomic profile comprises information regarding a metabolite biomarker, e.g., an oncometabolite. In some embodiments, the metabolite biomarker comprises one or more of a linolenic acid, glutamine, threonine, isoleucine, phospholipid, total choline-containing compound (tCho), phosphocholine, glucose, glycerophosphocholine, lactate, alanine, citrate, spermine, D-2-hydroxyglutarate, L-2-hydroxyglutarate, succinate, and fumarate. In some embodiments, the metabolic or metabolomic profile comprises information regarding one or more of glycolytic capacity, glucose metabolism, glutamine metabolism, glutaminolytic function, and lipidomics.

In some embodiments, the microbial profile comprises information regarding one or more microbe. Certain exemplary microbes include bacteria, viruses, and fungi. In some embodiments, the microbiome profile comprises one or more bacterial biomarkers. The bacterial biomarker may be one or more of Fusobacterium spp., Alphapapillomavirus genus, Proteobacteria, Pseudoxanthomonas, Actinobacteria, Saccharopolyspora, and Streptomyces. In some embodiments, the profile of the microbiome comprises a measure of the alpha-diversity of the bacterial community.

In some embodiments, the microbial profile comprises a viral nucleic acid. Certain exemplary viral nucleic acids include hepatitis B virus (HBV), human papilloma virus (HPV), human herpes virus, and Epstein-Barr virus (EBV).

In some embodiments, the immune cell profile comprises information regarding one or more of a population of B cells, T cells, neutral killer (NK) cells, neutrophils, eosinophils, basophils, monocytes, dendritic cells, macrophages, tumor-associated macrophages (TAM), and myeloid-derived suppressor cells (MDSC). In some embodiments, the T cell profile comprises information regarding one or more of a population of cytolytic T lymphocytes (CTL), regulatory T cells (Tregs), CD3+ T cells, CD4+ T cells, CD8+ T cells, and CD95+ T-helper cells. The immune cell profile may comprise information regarding T-cell receptor repertoire.

Any DNA profile, RNA profile, epigenetic profile, peptide, protein or proteomic profile, or metabolic or metabolomic profile described herein may comprise data from tumor-derived exosomes.

### Druggable Targets and Drugs

In some embodiments, any biomarker described herein may indicate one or more druggable targets. Certain exemplary druggable targets include a genetic variant; an RNA expression product of one or more genetic variants; a peptide or protein encoded by one or more genetic variants; a nucleic acid, peptide, or protein that shares a signaling pathway with the one or more genetic variants; a metabolite generated by one or more genetic variants; a metabolic pathway affected by the one or more genetic variants; and a microbial target.

Certain druggable targets that comprise a genetic variant are known in the art. Certain exemplary targetable genetic variants include epidermal growth factor receptor (EGFR; e.g., L858R, T790M), KRAS, NRAS, BRAF (e.g., V600E, V600K), phosphoinositide 3-kinase p110a (PIK3CA), phosphatase and tensin homolog (PTEN), human epidermal growth factor receptor 2 (HER2), c-Met, anaplastic lymphoma kinase (ALK), ROS1, and RET. In some embodiments, the druggable target comprises a signaling pathway. Certain exemplary signaling pathways include RAS/MAPK, PI3K/AKT/mTORC1, Notch, JAK/STAT, and VEGF/VEGFR.

In some embodiments, the druggable target is targeted by a small molecule drug. In some embodiments, the druggable target is targeted by a biologic drug (also termed biopharmaceutical or biological medical product). In some embodiments, the biologic drug comprises a vaccine, whole blood, blood component, allergenic, somatic cell, gene therapy, tissue, recombinant therapeutic protein, or a combination thereof. In some embodiments, the biologic drug comprises a sugar, peptide, protein, nucleic acid, or a combination thereof. In some embodiments, the biologic drug comprises living cells or tissues. In some embodiments, the biologic drug is isolated from living sources, e.g., humans, animals, plants, fungi, and/or microbes.

Certain targeted drug therapies are known in the art. Certain exemplary targeted drug therapies include drugs that target EGFR, ALK1, ROS1, tyrosine kinase receptors (TRKs), RET, fibroblast growth factor receptors (FGFRs), HER2, RAS, BRAF, tyrosine kinases, PI3K, mechanistic target of rapamycin (mTOR), Akt, Src homology-2 domain-containing protein tyrosine phosphatase-2 (SHP2), insulin-like growth factor 1 receptor (IGF1R), an immune checkpoint, neurotrophic tyrosine receptor kinase (NTRK) fusion, human growth factor (HGF)-c-Met, c-Kit, platelet-derived growth factor receptor (PDGFR), MEK/MAPK, cyclin-dependent kinase (CDK) 4/6, isocitrate dehydrogenase (IDH1 or IDH2), BRCA1/2 and ATM, estrogen receptor (ERa), aromatase, vascular endothelial growth factor receptor (VEGFR), poly (ADP-ribose) polymerase (PARP), and pyruvate kinase M2 (PKM2).

Certain exemplary EGFR-targeted therapies include cetuximab, panitumumab, gefitinib, Osimertinib, erlotinib, afatinib, and rociletinib. Certain exemplary ALK1-targeted therapies include crizotinib, alectinib, ceritinib, lorlatinib, and brigatinib. Certain exemplary ROS1-targeted therapies include crizotinib and entrectinib. Certain exemplary TRK-targeted therapies include entrectinib and Larotrectinib. Certain exemplary RET-targeted therapies include selpercatinib, pralsetinib, cabozantinib, and vandetanib. Certain exemplary FGFR-targeted therapies include erdafitinib, pemigatinib, infigratinib, rogaratinib, AZD4547, and dovitinib. Certain exemplary HER2-targeted therapies include trastuzumab, T-DM1, lapatinib, neratinib, tucatinib, pertuzumab, and antibody-drug conjugates (e.g., trastuzumab-emtansine and trastuzumab-deruxtecan). Certain exemplary RAS-targeted therapies include sotorasib, adagrasib, ARS3248, cetuximab, panitumumab, and JNJ-74699157. Certain exemplary BRAF-targeted therapies include encorafenib, cetuximab, binimetinib, vemurafenib, dabrafenib, trametinib, and cobimetinib. Certain exemplary tyrosine kinase inhibitor (TKI) therapies include lapatinib, tucatinib, imatinib, sunitinib, regorafenib, crenolanib, avapritinib, erlotinib, and gefitinib. Certain exemplary PI3K-targeted therapies include alpelisib, buparlisib and taselisib. Certain exemplary mTOR-targeted therapies include everolimus, temsirolimus, and sirolimus. Certain exemplary Akt-targeted therapies include capivasertib and ipatasertib. Certain exemplary immune-checkpoint inhibitors include pembrolizumab, nivolumab, cemiplimab, atezolizumab, avelumab, and durvalumab. Certain exemplary NTRK fusion-targeted therapies include entrectinib and Larotrectinib. Certain exemplary HGF-c-Met-targeted therapies include crizotinib, capmatinib, savolitinib, tepotinib, cabozantinib, foretinib, tivantinib, emibetuzumab, onartuzumab, ficlatuzumab, and rilotumumab. Certain exemplary c-Kit-targeted therapies include imatinib, sunitinib, regorafenib, sorafenib, dasatinib, and nilotinib. Certain exemplary PDGFR-targeted therapies include imatinib, dasatinib, sunitinib, regorafenib, crenolanib, and avapritinib. Certain exemplary MEK/MAPK-targeted therapies include trametinib, cobimetinib, selumetinib, and binimetinib. Certain exemplary CDK4/6-targeted therapies include Palbociclib, ribociclib, and abemaciclib. Certain exemplary IDH1-targeted therapies include ivosidenib. Certain exemplary IDH2-targeted therapies include enasidenib. Certain exemplary ERa-targeted therapies include fulvestrant, tamoxifen, and raloxifene. Certain exemplary aromatase-targeted therapies include anastrozole and exemestane. Certain exemplary VEGFR-targeted therapies include bevacizumab, ramucirumab, sorafenib, sunitinib, axitinib, tivozanib, pazopanib, regorafenib, and cediranib. Certain exemplary PARP-targeted therapies include olaparib, niraparib, rucaparib, talazoparib, and veliparib. Certain exemplary MSI high-targeted therapies include a checkpoint inhibitor, including those described herein.

In some embodiments, the methods, kits, and sets described herein are used to diagnose cancer in a child, adolescent, or adult subject. In some embodiments, the methods and sets described herein are used to diagnose, predict a druggable target for, confirm a druggable target for, select a treatment for, and/or treat any type of cancer. Certain exemplary cancers include lymphoblastic leukemia (ALL), acute myeloid leukemia (AML), adrenocortical carcinoma, anal cancer, bile duct cancer, bladder cancer, bone cancer, brain cancer, breast cancer, bronchial tumor, cardiac cancer, cervical cancer, chronic lymphocytic leukemia (CLL), chronic myelogenous leukemia (CML), chronic myeloproliferative neoplasm, colorectal cancer, endometrial cancer, esophageal cancer, intraocular melanoma, retinoblastoma, fallopian tube cancer, gallbladder cancer, gastric (stomach) cancer, gastrointestinal cancer, ovarian cancer, testicular cancer, gestational trophoblastic disease, hairy cell leukemia, head and neck cancer, hepatocellular carcinoma (HCC), islet cell tumor, pancreatic neuroendocrine tumor, kidney (renal cell) cancer, leukemia, lung cancer, non-small cell lung cancer, small cell lung cancer, liver cancer, lymphoma, Hodgkin lymphoma, non-Hodgkin lymphoma, melanoma, mesothelioma, multiple myeloma (MM), pancreatic cancer, paraganglioma, parathyroid cancer, penile cancer, pheochromocytoma, pituitary tumor, prostate cancer, rectal cancer, sarcoma, rhabdomyosarcoma, Kaposi sarcoma, Ewing sarcoma, osteosarcoma, skin cancer, small intestine cancer, thyroid cancer, urethral cancer, uterine cancer, vaginal cancer, and vulvar cancer. In some embodiments, the cancer is lung cancer. In some embodiments, the cancer is colorectal cancer.

1-9. (canceled)

10. A method of selecting a targeted cancer diagnostic test for a subject, comprising:
(a) performing or having performed a first test on a liquid biopsy sample of the subject to identify that the subject has a cancer, wherein the first test produces a first or second profile, optionally a first or second epigenetic profile, wherein the first profile is predictive of a first panel of one or more druggable targets in the subject and the second profile is predictive of a second panel of one or more druggable targets in the subject; and
(b) selecting a second test as the targeted cancer diagnostic test, wherein:
(i) if the first test produces the first profile, the second test assesses the presence or absence of the first panel of one or more druggable targets predicted by the first profile; and
(ii) if the first test produces the second profile, the second test assesses the presence or absence of the second panel of one or more druggable targets predicted by the first profile.

11. (canceled)

12. The method of claim 11, wherein the second test is performed on a liquid biopsy sample of the subject, optionally wherein the first and second tests are performed on different liquid biopsy samples of the subject.

13.-14. (canceled)

15. The method of claim 10, wherein the first test comprises a methylation assay, a nucleic acid assay (e.g., a genetic screen), a protein assay, a protein post-translational modification assay, a fragmentomics assay, a metabolomics assay, an RNA assay (e.g., a microRNA (miRNA) assay), a microbiome assay, an assay of one or more immune cell populations, or a combination thereof.

16. The method of claim 10, wherein the first test comprises a methylation assay, a nucleic acid assay (e.g., a genetic screen), a protein assay, a fragmentomics assay, or a combination thereof.

17. The method of claim 10, wherein the first test comprises a methylation assay.

18. The method of claim 10, wherein the profile produced by the first test comprises data on methylation state, chromatin compaction, histone modification, fragmentation patterns, topology, other epigenetic data, nucleic acid sequence, nucleic acid expression, protein translation, protein sequence, protein post-translational modification (e.g., glycosylation), metabolite presence, microbiome composition, immune state, or a combination thereof.

19. The method of claim 10, wherein the profile produced by the first test comprises data on methylation state, chromatin compaction, histone modification, fragmentation patterns, topology, other epigenetic data, or a combination thereof.

20. The method of claim 10, wherein the first test comprises multiple assays.

21. The method of claim 10, wherein the profile produced by the first test comprises data from multiple assays.

22. The method of claim 10, wherein the second test comprises a test for one or more genetic variants.

23. The method of claim 22, wherein the one or more druggable targets comprises the one or more genetic variants.

24. The method of claim 22, wherein the one or more druggable targets comprises a ribonucleic acid expression product of the one or more genetic variants.

25. The method of claim 22, wherein the one or more druggable targets comprises a peptide or protein encoded by the one or more genetic variants.

26. The method of claim 22, wherein the one or more druggable targets comprises a nucleic acid, peptide, or protein that shares a signaling pathway with the one or more genetic variants.

27. The method of claim 10, wherein the liquid biopsy sample comprises a blood sample.

28. The method of claim 10, wherein the liquid biopsy sample comprises cell-free DNA.

29.-45. (canceled)
